(19) United States
(12) Patent Application Publication
(10) Pub. No.: US 2026/0321381 A1
(43) Pub. Date: Oct. 15, 2026

(54) METHOD AND SYSTEM FOR PREDICTING PROTEIN BIOMARKER CONCENTRATION IN BIOLOGICAL FLUIDS VIA NON-PROTEIN BIOMARKER MEASUREMENT AND MACHINE LEARNING

(71) Applicant: Stephen Y. Chou, Princeton, NJ (US)

(72) Inventor: Stephen Y. Chou, Princeton, NJ (US)

(21) Appl. No.: 19/642,863

(22) Filed: Apr. 9, 2026

Related U.S. Application Data
(60) Provisional application No. 63/785,930, filed on Apr. 9, 2025.
(60) Provisional application No. 63/785,951, filed on Apr. 9, 2025.
(60) Provisional application No. 63/785,970, filed on Apr. 9, 2025.

(51) Int. Cl. G16H 20/10; A61B 5/145; G16B 40/20; G16H 10/60; G16H 40/67; G16H 50/20; G16H 50/70

(52) U.S. Cl. CPC G16H 20/10; A61B 5/14503; A61B 5/14532; A61B 5/14539; A61B 5/14546; G16B 40/20; G16H 10/60; G16H 40/67; G16H 50/20; G16H 50/70

(57) ABSTRACT

A method and system are disclosed for predicting the concentration of a targeted protein biomarker in a biological fluid of a subject without direct immunoassay-based protein measurement. Concentrations of a plurality of non-protein biomarkers are measured using continuously operable, miniaturizable sensors, and a trained machine learning model is applied to the measured concentrations to generate a predicted concentration of the targeted protein biomarker. The machine learning model is trained on a paired dataset of protein and non-protein biomarker measurements from an ensemble of biological fluid samples. The method is applicable to any biological fluid and to protein biomarkers across medical disciplines. In certain embodiments, the method is implemented in implanted, wearable, or point-of-care sensor platforms for continuous, real-time protein monitoring and closed-loop drug delivery.

### CROSS-REFERENCE. TO RELATED APPLICATIONS

The present application claims the benefit of U.S. Provisional Patent Application No. 63/785,930, filed Apr. 9, 2025, entitled “Detection and Monitoring of Ocular Protein in Tear”; U.S. Provisional Patent Application No. 63/785,951, filed Apr. 9, 2025, entitled “Punctum-Embedded Device and Its System for Ocular Biomarker Sensing, Diagnostic, Drug-Delivery, and Therapeutics”; and U.S. Provisional Patent Application No. 63/785,970, filed Apr. 9, 2025, entitled “Accurate Periocular Assay Under Imperfect Conditions,” the entire disclosures of which are incorporated herein by reference in their entirety.

### TECHNICAL FIELD

The present disclosure relates generally to medical diagnostics and biosensing. More particularly, the disclosure relates to methods and systems for predicting the concentration of a targeted protein biomarker in a biological fluid by measuring the concentrations of a plurality of non-protein biomarkers in the same biological fluid, without directly measuring the targeted protein, using a trained machine learning model and the prediction can be continuously in time. While tear fluid represents a preferred embodiment, the disclosure is broadly applicable to any biological fluid in which protein and non-protein biomarkers co-vary, including but not limited to blood, blood serum, blood plasma, urine, saliva, cerebrospinal fluid (CSF), sweat, interstitial fluid, synovial fluid, and other bodily fluids.

### BACKGROUND

### A. Protein Biomarkers in Biological Fluids and Need for Continuous Monitoring

Protein biomarkers in biological fluids serve as primary indicators of disease states across virtually every medical discipline. In neurology, amyloid-β and tau proteins in cerebrospinal fluid and, increasingly, in blood are emerging biomarkers for Alzheimer's disease. In ophthalmology, MMP-9, lactoferrin, lysozyme, and inflammatory cytokines in tear fluid reflect ocular surface disease and systemic conditions. In oncology, proteins such as prostate-specific antigen (PSA), carcinoembryonic antigen (CEA), cancer antigen 125 (CA-125), and HER2/neu are widely used for cancer screening and monitoring. In cardiovascular medicine, troponin I and T, B-type natriuretic peptide (BNP), and C-reactive protein (CRP) are key markers of cardiac injury and inflammation. In nephrology, cystatin C and kidney injury molecule-1 (KIM-1) indicate renal function. Across all these applications, the targeted protein biomarker is regarded as a primary, highly specific indicator of the relevant disease condition.

In monitoring and treatment a patient, there is a great need to continuously monitoring the concentration of a targeted protein in a biofluid. Such continuously protein monitoring can provide many vital information about the patient and diseases that cannot be provided by a single measurement. It can facilitate the diagnose of the patient's disease status. And it can enable automatic (e.g. AI driven) close-loop of monitoring, diagnostic, and therapeutics (drug treatment).

### B. Fundamental Limitations of Current Direct Protein Measurement

Limitation of Immunoassays: Each Sensor Is Single-Use Only and Not Suitable for Repeated or Continuous Protein Measurement Over Time. Immunoassay is the dominant method for quantifying specific proteins in biological fluids. The method exploits the highly specific and strong non-covalent binding between a target protein (antigen) and a cognate antibody. However, the high binding affinity between antibody and antigen—which confers specificity—simultaneously renders conventional immunoassay biosensors single-use: once the antibody-antigen complex forms, it is extremely difficult to dissociate, preventing sensor regeneration and precluding continuous or repeated measurement on the same sensor. This limitation is fundamental, not merely technological, as it arises from the thermodynamic stability of the antibody-antigen complex.

Specificity limitations of individual non-protein biomarkers: many non-protein biomarkers in biological fluids—including small molecules, metabolites, electrolyte ions, lipids, and nucleic acids—each can be measured, by a single sensor repeatedly or continuously. These non-protein biomarkers often change in concentration in response to disease conditions. However, each individual non-protein biomarker, measured in isolation, typically lacks a reliable one-to-one correspondence with the concentration of any specific protein biomarker, because the non-protein analytes are influenced by multiple concurrent physiological and environmental variables. They are thus classified as secondary (indirect) biomarkers with limited stand-alone diagnostic specificity.

### C. The Need for a New Approach

There is a broadly recognized unmet need for a method that: (1) enables continuous, repeated, or real-time monitoring of targeted protein biomarker concentrations; (2) is implementable on miniaturized sensor platforms compatible with wearable, implantable, or point-of-care devices; (3) does not require immunoassay-based protein capture; and (4) achieves clinically actionable prediction accuracy. The present disclosure addresses this unmet need by providing a machine learning-based method for predicting protein biomarker concentrations indirectly from non-protein biomarker measurements, applicable across biological fluids and medical domains.

### BRIEF SUMMARY

The present disclosure provides, in a first aspect, a method for predicting a concentration of a targeted protein biomarker in a biological fluid of a subject without directly measuring the concentration of the targeted protein biomarker in the biological fluid sample. The method comprises measuring, in a sample of biological fluid obtained from the subject, concentrations of a plurality of non-protein biomarkers, wherein the plurality of non-protein biomarkers comprises at least two distinct non-protein analytes; and applying a trained machine learning model to the measured concentrations of the plurality of non-protein biomarkers to generate a predicted concentration of the targeted protein biomarker. The trained machine learning model is trained on a dataset comprising, for each of a plurality of biological fluid samples, (i) a measured concentration of the targeted protein biomarker and (ii) measured concentrations of the plurality of non-protein biomarkers.

In certain embodiments of the first aspect, the concentration of each of the plurality of non-protein biomarkers is measured repeatedly by a sensor over time, and the trained machine learning model is applied repeatedly to predict the concentration of the targeted protein biomarker at each time point at which the concentrations of the plurality of non-protein biomarkers are measured.

In certain embodiments, the plurality of non-protein biomarkers comprises one or more analytes selected from the group consisting of electrolytes, ions, small molecules, metabolites, lipids, fatty acids, and nucleic acids. In certain embodiments, the plurality of non-protein biomarkers comprises at least three analytes selected from the group consisting of Na⁺, K⁺, Cl⁻, Ca²⁺, pH, osmolarity, glucose, lactate, urea, creatinine, and uric acid. In certain embodiments, the plurality of non-protein biomarkers comprises at least one electrolyte ion, at least one small molecule metabolite, and at least one analyte selected from the group consisting of pH and osmolarity.

In certain embodiments, the targeted protein biomarker is an ocular protein biomarker measured in tear fluid, selected from the group consisting of MMP-9, lactoferrin, lysozyme, lipocalin-1, secretory IgA, IL-1β, IL-6, IL-8, TNF-α, clusterin, annexin A1, tear-specific prealbumin, and MMP-3. In certain embodiments, the targeted protein biomarker is a blood-based protein biomarker selected from the group consisting of troponin I, troponin T, BNP, NT-proBNP, CRP, PSA, CEA, CA-125, AFP, HER2/neu, insulin, C-peptide, amyloid-β, tau, neurofilament light chain, GFAP, and IL-6. In certain embodiments, the targeted protein biomarker is a urinary protein biomarker selected from the group consisting of cystatin C, KIM-1, NGAL, microalbumin, and beta-2 microglobulin.

In certain embodiments, the biological fluid is selected from the group consisting of tear fluid, whole blood, blood serum, blood plasma, urine, saliva, cerebrospinal fluid, sweat, interstitial fluid, synovial fluid, pleural fluid, peritoneal fluid, amniotic fluid, nasal secretions, and combinations thereof.

In certain embodiments, the trained machine learning model comprises a model architecture selected from the group consisting of an artificial neural network, a deep neural network, a recurrent neural network, a support vector regression model, a random forest regression model, a gradient boosting model, a Gaussian process regression model, and an ensemble of two or more of the foregoing.

In certain embodiments, the method further comprises calculating, by an Error Risk Analysis (ERA) algorithm, a risk score for the predicted concentration representing a probability that a prediction error exceeds a predefined threshold; and discarding the predicted concentration and initiating a new measurement cycle when the risk score exceeds the predefined threshold.

In certain embodiments, the trained machine learning model is a personalized model that is trained or fine-tuned on biological fluid samples obtained from the subject, in addition to population-level training data, to improve prediction accuracy for the individual subject.

In certain embodiments, the predicted concentration of the targeted protein biomarker is used to generate a dosing instruction for delivery of a therapeutic agent to the subject, and the therapeutic agent is delivered by a drug delivery device in response to the dosing instruction.

The present disclosure provides, in a second aspect, a method of training a machine learning model for predicting a concentration of a targeted protein biomarker in a biological fluid. The method comprises obtaining an ensemble of biological fluid samples from a subject population; measuring, in each sample of the ensemble, the concentration of the targeted protein biomarker using a quantitative protein detection method; measuring, in each sample of the ensemble, the concentrations of a plurality of non-protein biomarkers, to create a paired training dataset; and training a machine learning model on the paired training dataset to learn a predictive relationship between the plurality of non-protein biomarker concentrations and the targeted protein biomarker concentration.

In certain embodiments of the second aspect, training the machine learning model further comprises applying a Nano-Enabled AI and Machine Learning (NEAM) approach, wherein intelligent nanostructures embedded in the sensor array provide physical and chemical condition-monitoring signals as additional input features, improving model accuracy and robustness.

The present disclosure provides, in a third aspect, a system for predicting a concentration of a targeted protein biomarker in a biological fluid without immunoassay-based protein measurement. The system comprises a sensor array configured to simultaneously measure concentrations of a plurality of non-protein biomarkers in a biological fluid, wherein the sensor array does not perform immunoassay-based protein capture; and a machine learning inference engine configured to receive the measured concentrations of the plurality of non-protein biomarkers and to output a predicted concentration of a targeted protein biomarker, wherein the machine learning inference engine executes a trained machine learning model trained on paired measurements of the targeted protein biomarker concentration and the plurality of non-protein biomarker concentrations obtained from an ensemble of biological fluid samples.

In certain embodiments of the third aspect, the sensor array comprises at least one ion-selective electrode (ISE) sensor, at least one enzymatic electrochemical sensor, and at least one sensor selected from the group consisting of a pH sensor and a conductometric osmometer. In certain embodiments, the sensor array is integrated within a miniaturized implantable or wearable device selected from the group consisting of a punctal plug configured for implantation in a lacrimal punctum, a subcutaneous implant, and a microneedle patch. In certain embodiments, the machine learning inference engine executes on a processor selected from the group consisting of an integrated circuit chip mounted on the sensor array device, an edge computing device in wireless communication with the sensor array, and a cloud computing platform. In certain embodiments, the system further comprises a drug delivery device configured to receive a dosing instruction generated based on the predicted protein biomarker concentration and to deliver a therapeutic agent to the subject in response to the dosing instruction, forming a closed-loop protein-guided therapeutic system.

The present disclosure provides, in a fourth aspect, a method for continuously monitoring a concentration of a targeted protein biomarker in a biological fluid of a subject over a monitoring period. The method comprises continuously or repeatedly measuring, using a sensor array in contact with or receiving a biological fluid from the subject, concentrations of a plurality of non-protein biomarkers at a plurality of time points during the monitoring period; applying, at each of the plurality of time points, a trained machine learning model to the measured non-protein biomarker concentrations to generate a predicted concentration of the targeted protein biomarker; and generating a longitudinal profile of predicted targeted protein biomarker concentration over the monitoring period. The sensor array does not perform immunoassay-based protein measurement, and continuous monitoring is enabled by the absence of antibody-antigen reagent consumption in the non-protein biomarker sensing steps.

In certain embodiments of the fourth aspect, the monitoring period is at least 24 hours and the non-protein biomarker concentrations are measured at intervals of one hour or less. In certain embodiments, a change in the predicted concentration of the targeted protein biomarker over the monitoring period is used to assess at least one of disease progression, therapeutic response, and need for clinical intervention. In certain embodiments, the biological fluid is tear fluid and the sensor array is embedded in a punctal plug implanted in a lacrimal punctum of an eye of the subject, enabling continuous, implanted monitoring of tear protein biomarker concentrations.

In one aspect, the present disclosure provides a method for predicting the concentration of a targeted protein biomarker in a biological fluid of a subject, comprising: measuring concentrations of a plurality of non-protein biomarkers in the biological fluid; and applying a trained machine learning model to the measured non-protein biomarker concentrations to generate a predicted concentration of the targeted protein biomarker; wherein the machine learning model is trained on a dataset of paired protein and non-protein biomarker measurements from an ensemble of biological fluid samples.

In another aspect, the disclosure provides a method of training a machine learning model for protein biomarker prediction, comprising: assembling a training dataset of paired measurements of a targeted protein biomarker and a plurality of non-protein biomarkers from an ensemble of biological fluid samples; and training a machine learning model on the training dataset to learn a predictive relationship between the non-protein biomarker concentrations and the targeted protein biomarker concentration.

In a further aspect, the disclosure provides a system for non-immunoassay-based protein biomarker monitoring, comprising: a sensor array configured to measure concentrations of a plurality of non-protein biomarkers in a biological fluid; and a machine learning inference engine configured to output a predicted concentration of a targeted protein biomarker from the measured non-protein biomarker concentrations.

In yet another aspect, the disclosure provides a closed-loop therapeutic system in which a predicted protein biomarker concentration, derived from non-protein biomarker measurements, drives automated delivery of a therapeutic agent to a subject.

The method and system of the disclosure are applicable to any biological fluid in which protein and non-protein biomarkers co-vary, and to protein biomarkers associated with any disease or physiological condition. Preferred embodiments include, but are not limited to, applications in ophthalmology (tear fluid), oncology (blood/serum), nephrology (urine/blood), neurology (CSF/blood), cardiology (blood/serum), and endocrinology (blood/interstitial fluid/sweat).

### BRIEF DESCRIPTION OF THE DRAWINGS

FIG. 1 is a schematic overview of the method of the disclosure illustrating an embodiment of the method of this disclosure for predicting a concentration of a targeted protein biomarker in a biological fluid of a subject without directly measuring the targeted protein biomarker.

FIG. 2 is a flowchart of the training phase, including biological fluid sample collection, paired protein and non-protein biomarker measurement, training dataset assembly, and machine learning model training and validation.

### DETAILED DESCRIPTION

The following detailed description is presented to enable any person skilled in the art to make and use the disclosure. Specific details are set forth to provide a thorough understanding of the present disclosure. However, it will be apparent to one skilled in the art that these specific details are not required in every case.

As used herein, “biological fluid” means any fluid of biological origin obtainable from a subject, including without limitation: tear fluid, whole blood, blood serum, blood plasma, urine, saliva, cerebrospinal fluid, sweat, interstitial fluid, synovial fluid, peritoneal fluid, pleural fluid, amniotic fluid, breast milk, and nasal secretions. “Targeted protein biomarker” means a specific protein molecule present in a biological fluid whose concentration is diagnostically, prognostically, or therapeutically relevant to a disease, condition, or physiological state. “Non-protein biomarker” means any biochemical analyte measurable in a biological fluid that is not itself a protein, including without limitation: electrolyte ions, pH, osmolarity, small organic molecules, metabolites, lipids, fatty acids, nucleic acids, hormones, and reactive oxygen species. “Continuous monitoring” means repeated or real-time measurement at intervals sufficient to track physiologically meaningful changes over time.

One insight of the present disclosure is that while individual non-protein biomarkers in a biological fluid each lack a reliable one-to-one predictive relationship with a specific protein biomarker, the ensemble of multiple non-protein biomarkers collectively encodes sufficient multivariate information—through their joint pattern of co-variation with the protein target—to enable a trained machine learning model to predict the targeted protein biomarker concentration with clinically actionable accuracy. This indirect prediction approach fundamentally decouples the accuracy of protein biomarker monitoring from the constraints of immunoassay technology, and is therefore continuously operable, miniaturizable, and compatible with electrochemical sensor platforms.

The proposed technique rests on the biological principle that protein and non-protein analytes in biological fluids are not biochemically independent: they are co-regulated by shared physiological processes—including disease-driven inflammation, metabolic dysregulation, cellular damage, immune activation, and organ dysfunction—that simultaneously alter the concentrations of both protein and non-protein species in predictable, learnable patterns. A machine learning model, trained on sufficiently large and diverse paired datasets, can capture these multivariate co-variation patterns and exploit them for prediction.

The proposed approach comprises two phases: (1) a training phase, in which a machine learning model is trained on paired measurements of protein and non-protein biomarkers; and (2) an inference phase, in which only non-protein biomarker measurements are made and the trained model generates predicted protein concentrations. These two phases may be executed at different times, locations, and on different hardware.

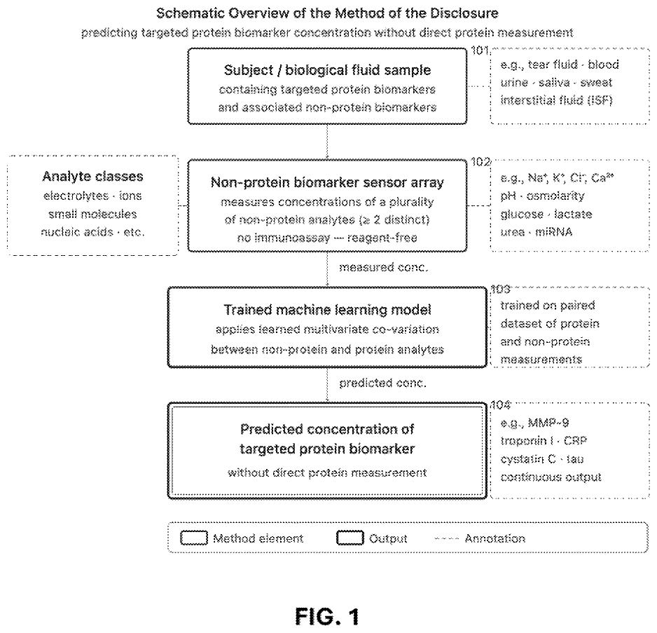

FIG. 1 is a schematic overview of the method of the disclosure illustrating the method of Claim 1 for predicting a concentration of a targeted protein biomarker in a biological fluid of a subject without directly measuring the targeted protein biomarker. Step 101 depicts obtaining a biological fluid sample from the subject, including without limitation tear fluid, blood, urine, saliva, sweat, and interstitial fluid. Step 102 depicts measuring, in the biological fluid sample, the concentrations of a plurality of non-protein biomarkers comprising at least two distinct non-protein analytes, wherein the non-protein biomarkers are selected from analyte classes including electrolytes, ions, small molecules, nucleic acids, and related analyte classes, with representative examples including Na⁺, K⁺, Cl⁻, Ca²⁺, pH, osmolarity, glucose, lactate, urea, and miRNA. Step 103 depicts applying a trained machine learning model to the measured non-protein biomarker concentrations, wherein the trained machine learning model was trained on a paired dataset of protein and non-protein biomarker measurements. Step 104 depicts the output of the method: a predicted concentration of the targeted protein biomarker, including without limitation MMP-9, troponin I, CRP, cystatin C, and tau, generated without direct protein measurement and suitable for continuous real-time output.

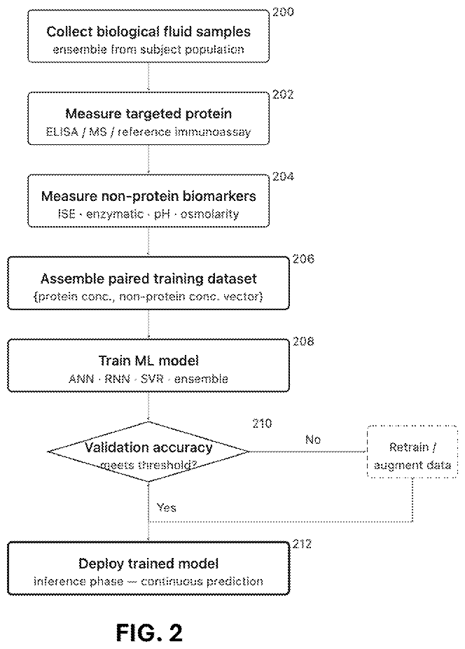

FIG. 2 is a flowchart of the training phase, including biological fluid sample collection, paired protein and non-protein biomarker measurement, training dataset assembly, and machine learning model training and validation. Step 200 depicts collecting an ensemble of biological fluid samples from a subject population. Step 202 depicts measuring, in each sample of the ensemble, the concentration of the targeted protein biomarker using a quantitative reference protein detection method, such as enzyme-linked immunosorbent assay (ELISA), mass spectrometry, or immunoassay. Step 204 depicts measuring, in each sample of the ensemble, the concentrations of a plurality of non-protein biomarkers using appropriate sensor technologies. Step 206 depicts assembling a paired training dataset comprising, for each sample, the measured protein biomarker concentration and the measured non-protein biomarker concentration vector. Step 208 depicts training a machine learning model on the paired training dataset to learn the predictive relationship between the non-protein biomarker concentrations and the targeted protein biomarker concentration. Step 210 depicts a validation decision point at which the prediction accuracy of the trained model is evaluated against a predefined threshold; if the accuracy threshold is not met, the model is retrained or the training dataset is augmented as indicated by the feedback loop returning to step 208. Step 212 depicts deployment of the validated trained model for use in the inference phase, enabling continuous, real-time prediction of the targeted protein biomarker concentration from non-protein biomarker measurements as described in FIG. 1.

### Biological Fluid Embodiments

The method and system of the invention are applicable to any biological fluid. The following non-limiting embodiments illustrate the breadth of application:

### A. Tear Fluid (Ophthalmology)

Tear fluid is a particularly preferred embodiment. Tear protein biomarkers including MMP-9, lactoferrin, lysozyme, lipocalin-1, secretory IgA, and inflammatory cytokines (IL-1β, IL-6, IL-8, TNF-α) are primary biomarkers for dry eye disease (DED), ocular surface inflammation, meibomian gland dysfunction, and other ophthalmic conditions. Tear non-protein biomarkers including osmolarity, Na⁺, K⁺, Cl⁻, pH, glucose, and lactate are measurable by miniaturized electrochemical sensors compatible with implantable punctal plug devices. This embodiment enables continuous, implanted MMP-9 or lactoferrin monitoring—not achievable by any prior art—and supports closed-loop anti-inflammatory drug delivery for DED management.

### B. Blood, Serum, and Plasma (Oncology, Cardiology, General Medicine)

In blood-based embodiments, the targeted protein biomarkers include cancer biomarkers (PSA, CEA, CA-125, AFP, HER2/neu), cardiac biomarkers (troponin I, troponin T, BNP, NT-proBNP, CRP), metabolic biomarkers (insulin, leptin, adiponectin), inflammatory biomarkers (IL-6, TNF-α, ferritin), and others. Non-protein biomarkers in blood that co-vary with these proteins include glucose, lactate, urea, creatinine, uric acid, pH, electrolytes (Na⁺, K⁺, Cl⁻, Ca²⁺), lipids (cholesterol, triglycerides, LDL, HDL), and microRNAs. Wearable or implantable electrochemical sensors capable of continuous blood or interstitial fluid monitoring (e.g., continuous glucose monitors, multi-analyte biosensors) provide the non-protein measurement inputs for the model.

### C. Urine (Nephrology, Oncology, Metabolic Disease)

In urine-based embodiments, targeted proteins include cystatin C, KIM-1, NGAL (neutrophil gelatinase-associated lipocalin), albumin (microalbuminuria), and beta-2 microglobulin, which are markers of renal function and damage. Non-protein biomarkers in urine include creatinine, pH, osmolarity, glucose, urea, electrolytes, and ketones, all measurable by standard electrochemical and colorimetric sensors. Prediction of urinary protein biomarkers from non-protein urine analytes enables continuous renal function monitoring through wearable or smart-toilet biosensing platforms.

### D. Saliva (Dentistry, Oncology, Systemic Disease Monitoring)

In saliva-based embodiments, targeted proteins include salivary alpha-amylase, IL-8, IL-6, TNF-α, MMP-8, and cancer biomarkers. Salivary non-protein biomarkers include pH, nitrite, glucose, lactate, uric acid, and electrolytes. Saliva-based protein prediction is particularly attractive for point-of-care diagnostics because saliva collection is non-invasive and suitable for frequent self-sampling.

### E. Cerebrospinal Fluid (Neurology)

In CSF-based embodiments, targeted proteins include amyloid-β 42, phosphorylated tau (p-tau), total tau, neurofilament light chain (NfL), and alpha-synuclein, which are primary biomarkers for Alzheimer's disease, Parkinson's disease, and other neurodegenerative conditions. Non-protein biomarkers in CSF include glucose, lactate, pH, and various metabolites. Prediction of CSF protein biomarker concentrations from non-protein analytes could reduce the need for invasive lumbar puncture by enabling inference from less invasive biological fluid measurements.

### F. Sweat and Interstitial Fluid (Wearable Sensing)

In sweat and interstitial fluid embodiments, targeted proteins include cytokines (IL-6, TNF-α), cortisol (a hormone sometimes measurable by immunoassay in sweat), and other stress and immune response proteins. Non-protein biomarkers in sweat include Na⁺, K⁺, Cl⁻, lactate, glucose, uric acid, pH, and ammonia, all of which are measurable by wearable electrochemical skin-surface sensors. Prediction of protein biomarker concentrations in sweat enables fully non-invasive, wearable continuous health monitoring.

### Non-Protein Biomarker Classes

The plurality of non-protein biomarkers used as predictive input features for the machine learning model may be selected from, but are not limited to, the following classes (applicable across biological fluids):
(a) Electrolytes and Ions: Sodium (Na⁺), potassium (K⁺), chloride (Cl⁻), calcium (Ca²⁺), magnesium (Mg²⁺), bicarbonate (HCO₃⁻), phosphate (PO₄⁻), and ammonium (NH₄⁺), measurable by ion-selective electrode (ISE) sensors.
(b) pH and Osmolarity: Fluid pH (pH-sensitive electrodes) and osmolarity (conductimetric osmometry), applicable in all biological fluids.
(c) Small Molecules and Metabolites: Glucose, lactate, urea, creatinine, uric acid, ascorbic acid, glutathione, pyruvate, ketones, and others, measurable by enzyme-based electrochemical sensors (EAEC).
(d) Lipids and Fatty Acids: Cholesterol, triglycerides, free fatty acids, phospholipids, sphingolipids, eicosanoids, and LDL/HDL fractions.
(e) Nucleic Acids: MicroRNAs (miRNAs), cell-free DNA (cfDNA), and other circulating nucleic acids, measurable by electrochemical hybridization sensors.
(f) Hormones and Signaling Molecules: Cortisol, melatonin, and other non-protein hormones or small-molecule signaling agents detectable in biological fluids.
(g) Reactive Oxygen Species (ROS): Hydrogen peroxide, superoxide, and other oxidative stress markers.
(h) Other Physical Parameters: Temperature, viscosity, and optical turbidity of the biological fluid, when measurable and correlatable with protein biomarker concentration.

### Targeted Protein Biomarkers

The method is applicable to any protein biomarker present in a biological fluid for which a predictive relationship with measurable non-protein biomarkers can be learned from a training dataset. Non-limiting examples organized by medical domain include:

### A. Ophthalmology (Tear Fluid)

MMP-9, lactoferrin, lysozyme, lipocalin-1, secretory IgA, tear-specific prealbumin (TSPA), clusterin, annexin A1, serum albumin, transferrin, zinc-α2-glycoprotein, secretory phospholipase A2, cystatin C, IL-1β, IL-6, IL-8, TNF-α, and MMP-3.

### B. Oncology (Blood/Serum)

PSA (prostate cancer), CEA (colorectal, lung, breast cancer), CA-125 (ovarian cancer), AFP (hepatocellular carcinoma), HER2/neu (breast cancer), CA 19-9 (pancreatic cancer), VEGF, and circulating tumor-associated proteins.

### C. Cardiology (Blood/Serum)

Troponin I, troponin T, BNP, NT-proBNP, CRP, D-dimer, myoglobin, and FABP (fatty acid-binding protein).

### D. Nephrology (Urine/Blood)

Cystatin C, KIM-1, NGAL, microalbumin, beta-2 microglobulin, and TIMP-2.

### E. Neurology (CSF/Blood)

Amyloid-β 40, amyloid-β 42, phosphorylated tau (p-tau 181, p-tau 217), total tau, neurofilament light chain (NfL), alpha-synuclein, and GFAP (glial fibrillary acidic protein).

### F. Endocrinology and Metabolic Disease (Blood/Interstitial Fluid)

Insulin, C-peptide, leptin, adiponectin, GLP-1, IGF-1, and thyroid-stimulating hormone (TSH).

### G. Infectious Disease and Immunology (Multiple Fluids)

CRP, procalcitonin (PCT), ferritin, IL-6, and pathogen-specific antigens detectable in blood, saliva, or other fluids.

### Training Phase: Dataset Construction and Model Training

### A. Training Dataset Assembly

An ensemble of biological fluid samples is collected from a subject population that preferably spans a range of disease conditions, disease severities, treatment states, demographic variables, and other clinically relevant covariates. For each sample in the ensemble, the following measurements are performed: (i) the concentration of the targeted protein biomarker, measured by any available quantitative reference method (e.g., ELISA, mass spectrometry, immunoassay, optical biosensor); and (ii) the concentrations of a plurality of non-protein biomarkers, measured by appropriate sensor technologies. The paired measurements constitute the training dataset. The size of the training ensemble is determined by model complexity and the number of non-protein features; in preferred embodiments, the ensemble comprises at least several hundred paired samples, and preferably several thousand or more.

### B. Machine Learning Model Training

The training dataset is used to train a machine learning model to learn the predictive relationship between the non-protein biomarker concentration vector and the targeted protein biomarker concentration. The model is not limited to any specific algorithm; suitable model architectures include: artificial neural networks (ANN); deep neural networks (DNN); convolutional neural networks (CNN) where temporal or spatial structure in the data is present; recurrent neural networks (RNN) or long short-term memory networks (LSTM) for time-series monitoring data; support vector regression (SVR); random forest regression; gradient boosting methods (XGBoost, LightGBM, CatBoost); Gaussian process regression; and ensemble methods. Model selection and hyperparameter optimization are performed using cross-validation on held-out subsets of the training data.

Where biological fluid sample collection is from a specific anatomical site (e.g., a specific punctum, a specific vascular access point), transfer learning may be applied to adapt a population-trained model to individual subjects using smaller subject-specific calibration datasets, improving personalized prediction accuracy.

Nano-Enabled AI and ML (NEAM)—wherein intelligent nanostructures embedded within the sensor array provide physical and chemical condition monitoring signals beyond standard sensor outputs—may be applied to further improve model robustness by enabling dynamic compensation for sensor drift, matrix effects, and environmental perturbations during both training and inference.

An Error Risk Analysis (ERA) module may be incorporated to calculate a risk score for each predicted output, representing the probability that the prediction exceeds a predefined accuracy error threshold. Predictions with risk scores above the threshold are flagged and, where applicable, a new measurement is automatically initiated. ERA prevents inaccurate predictions from being acted upon in diagnostic or therapeutic decisions.

### Inference Phase: Real-Time Protein Prediction

In the inference phase, the trained machine learning model is deployed in a clinical, point-of-care, or continuous monitoring context. Non-protein biomarker concentrations are measured in the biological fluid of a subject—as a single-time-point measurement or continuously—using a sensor array. The measured non-protein concentration vector is input to the trained model, which outputs a predicted concentration of the targeted protein biomarker. Because non-protein biomarker sensors do not rely on antibody-antigen reactions, they can be regenerated or operated continuously over extended periods, enabling continuous protein prediction without sensor replacement.

The inference computation may be executed locally on an onboard microprocessor or integrated circuit chip (in implanted or wearable devices), on an edge computing device (e.g., smartphone), or on a cloud computing platform, depending on the computational resources available and the latency requirements of the application.

### System Embodiments

### A. Implanted Punctal Plug System (Tear Fluid)

In a preferred embodiment for ophthalmic applications, the sensor array is integrated within a miniaturized punctal plug implanted in the lacrimal punctum of the eye. The sensor array continuously measures non-protein biomarker concentrations in the tear fluid flowing through the plug. Measurements are wirelessly transmitted to an external station, where the trained machine learning model generates a continuous stream of predicted protein biomarker concentrations. This embodiment is described in detail in co-pending U.S. Patent Application No. 63/785,951 (i-NanoOcuCare), incorporated herein by reference.

### B. Wearable Skin-Surface Sensor System

In a wearable embodiment, the sensor array is integrated into a skin-worn patch, wristband, or other wearable device that contacts sweat or measures interstitial fluid via minimally invasive microneedle arrays. The sensor array continuously measures non-protein biomarkers in sweat or interstitial fluid. The trained model predicts the concentration of a targeted protein biomarker from these measurements, enabling continuous, non-invasive protein monitoring in an ambulatory setting.

### C. Point-of-Care System

In a point-of-care embodiment, a biological fluid sample (blood, urine, saliva, tear) is introduced to a disposable sensor cartridge. The cartridge simultaneously measures the concentrations of the plurality of non-protein biomarkers and transmits the results to a handheld reader or smartphone application, which executes the trained model inference and displays the predicted protein biomarker concentration. This embodiment provides low-cost, rapid protein biomarker assessment without laboratory immunoassay.

### D. Closed-Loop Therapeutic System

In a closed-loop embodiment, the predicted protein biomarker concentration is input to a therapeutic algorithm that determines dosing instructions (dose amount and timing) for a drug delivery device, which delivers a therapeutic agent in response. The drug delivery device may be co-located with the sensing device (e.g., in the same or contralateral punctal plug for ophthalmic applications) or may be a separate implanted, wearable, or external device. Examples include: closed-loop anti-inflammatory drug delivery for DED guided by predicted MMP-9; closed-loop insulin delivery guided by predicted C-peptide or insulin; and closed-loop immunotherapy adjustment guided by predicted cytokine levels.

### VIII. Non-Limiting Examples of Advantages Over Conventional Techniques

Universality: The method is applicable to any biological fluid and any protein biomarker for which non-protein co-variates exist—a near-universal condition in biological systems driven by the shared physiological processes that regulate both protein and non-protein analytes.
Continuous Operation: The sensor types used for non-protein measurement (ISE, EAEC, pH, conductimetric) do not consume antibody reagents and can operate continuously, enabling persistent protein monitoring without sensor replacement or immunoassay replenishment.
Miniaturization: All required sensor types are electrochemical and do not require optical components, enabling integration in implanted, wearable, and point-of-care devices at scales impossible for conventional immunoassay systems.
Regulatory Flexibility: The method avoids the regulatory bottleneck of electrochemical immunoassays by relying entirely on established, non-immunoassay electrochemical sensing modalities.
Personalization: The machine learning model can be fine-tuned on individual patient data to improve personalized prediction accuracy, enabling increasingly precise monitoring as patient-specific data accumulates.
Extensibility: A single sensor array hardware platform, with appropriate model training, can predict multiple different protein biomarkers, amortizing hardware development cost across multiple clinical applications.

### Electrolytes and Ions

Electrolytes and ions—including sodium (Na⁺), potassium (K⁺), chloride (Cl⁻), calcium (Ca²⁺), and magnesium (Mg²⁺)—are continuously measured using ion-selective electrodes (ISEs) integrated onto the sensor chip. Each ISE comprises an ion-selective membrane formulated with a specific ionophore that selectively binds the target ion, generating a Nernstian potential proportional to the ion's activity in the biological fluid. For continuous monitoring, an array of ISEs is maintained in contact with the fluid, with periodic re-zeroing performed against an on-chip reference electrode to correct for baseline drift. Multiplexed readout electronics sample each electrode at defined time intervals, and the resulting time-series data are passed to the machine learning model for real-time analysis.

In further embodiments, electrolyte and ion concentrations are measured using optical sensors in which ion-selective fluorescent dyes or chromoionophores are embedded in a polymer film deposited on or adjacent to the sensor chip. Upon ion binding, the dye or chromoionophore undergoes a shift in fluorescence emission wavelength or intensity proportional to the ion activity in the biological fluid; this optical signal is detected by an on-chip photodetector integrated with the sensor array. Such optical ion sensors offer the advantage of electrical isolation from the sample, reducing interference from electroactive species and enabling operation in electrically noisy environments.

In additional embodiments, ion concentrations are measured using ion-sensitive field-effect transistors (ISFETs), in which the ion-selective membrane is disposed directly on or in proximity to the gate dielectric of a field-effect transistor. Ion activity at the gate surface modulates the threshold voltage and drain current of the transistor in a Nernst-dependent manner, providing a direct electrical readout of ion concentration. ISFETs are particularly well suited for miniaturization, low-power continuous operation, and integration into large-scale sensor arrays on a single chip.

In yet further embodiments, ion concentrations are measured using surface acoustic wave (SAW) sensors coated with ion-selective films. Acoustic waves propagate along the surface of a piezoelectric substrate; binding of target ions to the ion-selective coating increases the effective mass and alters the mechanical properties of the surface layer, producing a shift in the acoustic wave phase velocity and resonance frequency that is proportional to the ion concentration. SAW sensors are compatible with continuous, reagent-free operation and can be fabricated in compact array configurations.

### Small Molecules and Metabolites

Small molecules and metabolites—such as glucose, lactate, urea, creatinine, and ascorbic acid—are continuously monitored using enzymatic electrochemical sensors. In these sensors, an enzyme specific to the target analyte (e.g., glucose oxidase for glucose, lactate oxidase for lactate) is immobilized in a thin film directly on a working electrode. The enzymatic reaction generates a redox product, typically hydrogen peroxide, whose oxidation or reduction at the electrode surface produces a measurable amperometric current proportional to the analyte concentration. To enable continuous operation, the enzyme layer is encapsulated within a diffusion-limiting membrane that controls analyte flux, prevents enzyme leaching, and reduces interference from electroactive species present in the biological fluid. Continuous amperometric measurements are acquired at fixed intervals and logged as time-series signals for downstream processing.

In further embodiments, small molecule and metabolite concentrations are measured using optical sensors based on fluorescent boronic acid derivatives or competitive binding assay formats. Boronic acid groups form reversible covalent bonds with diol-containing analytes such as glucose and related small molecules; the resulting change in the electronic environment of the boronic acid-fluorophore conjugate produces a concentration-dependent shift in fluorescence intensity or lifetime that is detected by an on-chip photodetector. In competitive binding assay configurations, the target analyte displaces a fluorescently labeled competitor from a receptor, generating a ratiometric fluorescence signal proportional to analyte concentration.

In additional embodiments, small molecules and metabolites are continuously detected using affinity-based sensors in which aptamers or antibody fragments serve as recognition elements and are conjugated to fluorescent or plasmonic reporters. In fluorescence-based configurations, conformational changes in the aptamer or antibody fragment upon target binding alter the proximity of a fluorophore-quencher pair, producing a concentration-dependent fluorescence signal. In surface-enhanced Raman spectroscopy (SERS)-based configurations, the recognition element localizes the target analyte within the near-field of metallic nanostructures—gold or silver nanoparticles or nanostructured surfaces—that dramatically amplify the Raman scattering cross-section of the bound analyte, enabling label-free or minimally labeled continuous detection with molecular specificity.

In yet further embodiments, small molecule and metabolite concentrations are measured by mid-infrared (MIR) absorption spectroscopy, in which the biological fluid sample is interrogated by infrared radiation and each target analyte absorbs at characteristic molecular vibrational frequencies. The absorbed intensity at each characteristic wavelength is proportional to the analyte concentration in accordance with the Beer-Lambert law, enabling direct, reagent-free quantification without enzymatic or chemical reactions. Attenuated total reflectance (ATR) configurations, in which an evanescent infrared field penetrates only a thin layer of the biological fluid at the surface of an internal reflection element, are particularly well suited for continuous, on-chip measurement with minimal sample preparation.

### Lipids and Fatty Acids

Lipids and fatty acids are continuously measured using electrochemical aptasensors or molecularly imprinted polymer (MIP)-based sensors integrated on the chip. Aptamers or MIP recognition elements with high affinity and selectivity for target lipid species—such as free fatty acids, lysophospholipids, or cholesterol—are immobilized on the electrode surface. Binding of the target molecule induces a conformational change or a steric effect that alters the interfacial electron-transfer kinetics, which is detected as a change in impedance or voltammetric current via electrochemical impedance spectroscopy (EIS) or square-wave voltammetry (SWV). Because lipid concentrations in biological fluids such as tears are typically low, signal amplification strategies—including redox-active reporter molecules and nanostructured electrode surfaces—are employed to achieve the required sensitivity. Continuous monitoring is achieved by performing repeated measurement cycles at defined time intervals while applying regeneration pulses between cycles to restore the sensing surface.

### Nucleic Acids

Nucleic acids—including cell-free DNA (cfDNA), microRNA (miRNA), and messenger RNA (mRNA) fragments present in the biological fluid—are continuously monitored using electrochemical nucleic acid biosensors. Single-stranded DNA or RNA probe sequences complementary to the target nucleic acid are covalently immobilized on gold or carbon electrode surfaces. Hybridization of the target strand to the surface-bound probe produces a measurable change in the electrochemical signal, detected via differential pulse voltammetry (DPV) or EIS. To enable continuous and repeated measurements in a flowing sample, a regeneration step using a mild denaturing buffer or thermal pulse is applied periodically to dissociate hybridized duplexes and restore the probe layer. Alternatively, rolling circle amplification (RCA) or other isothermal amplification schemes may be incorporated on-chip to amplify trace nucleic acid signals prior to electrochemical detection, thereby improving sensitivity for the low concentrations typically encountered in biological fluids such as tears.

### Additional Embodiments

### A. Broader Method Embodiments

In certain embodiments, the inventive concept extends beyond the prediction of protein biomarkers specifically to encompass the prediction of any targeted analyte in a biological fluid from measurements of a plurality of secondary analytes that are distinct from the targeted analyte. In such embodiments, the method comprises measuring, in a sample of biological fluid obtained from a subject, concentrations of a plurality of secondary analytes that are not the targeted analyte, and applying a trained predictive model to the measured concentrations to generate a predicted concentration of the targeted analyte. The trained predictive model is trained on a dataset comprising paired measurements of the targeted analyte concentration and the plurality of secondary analyte concentrations from an ensemble of biological fluid samples. This generalized formulation encompasses any combination of analyte classes—including protein-from-non-protein, metabolite-from-ion, or nucleic-acid-from-metabolite prediction—and is not limited to the protein biomarker embodiments described above.

In certain embodiments, the method is further generalized to the inference of a concentration of a first biological analyte in a biological fluid from measurements of a plurality of second biological analytes in the same biological fluid, wherein the plurality of second biological analytes are distinct in chemical class from the first biological analyte. The trained machine learning model in such embodiments exploits multivariate co-variation between the first biological analyte and the plurality of second biological analytes that arises from shared physiological regulation—including disease-driven inflammation, metabolic dysregulation, cellular damage, immune activation, and organ dysfunction—that simultaneously modulates the concentrations of analytes across chemical classes in predictable, learnable patterns.

In certain embodiments, the method is implemented as a computer-implemented method for continuous protein biomarker monitoring without immunoassay. In such embodiments, the method comprises receiving, from a sensor array in contact with a biological fluid of a subject, a time-series of concentration measurements of a plurality of non-protein analytes; applying, at each of a plurality of time points in the time-series, a trained machine learning model to the non-protein analyte concentration measurements at that time point to generate a predicted protein biomarker concentration; and outputting a continuous stream of predicted protein biomarker concentrations over time. In all such embodiments, the sensor array does not employ antibody-antigen binding for any measurement, thereby enabling continuous, reagent-free operation not achievable by conventional immunoassay-based sensor platforms.

### B. Sensor Array and Hardware Embodiments

In certain embodiments, the invention provides a sensor array for continuous non-protein biomarker measurement in a biological fluid. The sensor array comprises a substrate; a plurality of electrochemical sensing elements disposed on the substrate, each sensing element configured to continuously measure a concentration of a distinct non-protein analyte in a biological fluid in contact with the substrate; a reference electrode disposed on the substrate and shared by the plurality of electrochemical sensing elements; a multiplexed signal acquisition circuit configured to sample each of the plurality of electrochemical sensing elements at defined time intervals; and an output interface configured to transmit the sampled concentration measurements to a machine learning inference engine. In certain such embodiments, the plurality of electrochemical sensing elements comprises at least one ion-selective electrode (ISE) for measuring an electrolyte ion concentration, at least one enzymatic electrochemical sensor for measuring a metabolite concentration, and at least one potentiometric pH sensor.

In certain embodiments, the substrate of the sensor array is configured for implantation within a punctal plug, and the sensor array has a form factor compatible with implantation in a human lacrimal punctum. In certain embodiments, the sensor array further comprises one or more intelligent nanostructures disposed on at least one sensing element surface, wherein the intelligent nanostructures provide physical and chemical condition-monitoring signals indicative of sensor operating conditions, sample matrix quality, or both, in accordance with the Nano-Enabled AI and Machine Learning (NEAM) approach described herein. In certain embodiments, the sensor array further comprises a microfluidic channel configured to deliver biological fluid to the plurality of electrochemical sensing elements, and at least one microfluidic pump configured to remove stale biological fluid from, and replenish fresh biological fluid to, the sensing elements at defined intervals, thereby ensuring that the non-protein biomarker measurements reflect the current physiological state of the subject rather than a stale or degraded sample.

In certain embodiments, the invention provides a miniaturized implantable biosensing device comprising a housing configured for implantation at an anatomical site in contact with a biological fluid; a sensor array disposed within the housing and configured to continuously measure concentrations of a plurality of non-protein biomarkers in the biological fluid; an integrated circuit disposed within the housing and configured to execute a trained machine learning model that generates a predicted concentration of a targeted protein biomarker from the measured non-protein biomarker concentrations; and a wireless communication interface disposed within the housing and configured to transmit the predicted protein biomarker concentration to an external device. In a particularly preferred embodiment, the housing is a punctal plug configured for implantation in a lacrimal punctum of a human eye, and the biological fluid is tear fluid, enabling continuous, implanted, wireless monitoring of tear protein biomarker concentrations as described above.

### C. OPNA Method Embodiments

In certain embodiments directed to ophthalmic applications, the invention is practiced as a method of Ocular Protein-detection via Non-protein biomarkers and Artificial Intelligence, herein referred to as OPNA. In OPNA embodiments, the method comprises continuously measuring, using a sensor array in fluidic contact with tear fluid of a subject, concentrations of a plurality of non-protein tear biomarkers; and applying a trained artificial intelligence model to the measured non-protein tear biomarker concentrations to generate a predicted concentration of an ocular protein biomarker, wherein the sensor array does not perform immunoassay-based protein capture. In certain OPNA embodiments, the plurality of non-protein tear biomarkers comprises two or more analytes selected from the group consisting of osmolarity, Na⁺, K⁺, Cl⁻, Ca²⁺, pH, glucose, lactate, urea, lysozyme activity, and lipid concentration. In a particularly preferred OPNA embodiment, the ocular protein biomarker is MMP-9, and the predicted MMP-9 concentration is used to diagnose dry eye disease, monitor dry eye disease severity, or guide delivery of an anti-inflammatory therapeutic agent to the ocular surface. The OPNA method represents a paradigm shift in ocular diagnostics, enabling for the first time continuous, implanted monitoring of a tear protein biomarker without direct immunoassay measurement, and supporting closed-loop anti-inflammatory therapy for dry eye disease management.

### D. Training Method and Model Personalization Embodiments

In certain embodiments, the invention provides a method of constructing a training dataset for the machine learning model described herein. The method comprises collecting an ensemble of biological fluid samples from a subject population spanning a plurality of disease conditions, disease severities, and demographic variables; measuring, in each sample of the ensemble using a quantitative reference protein detection method, a concentration of a targeted protein biomarker; measuring, in each sample of the ensemble using a plurality of non-immunoassay sensors, concentrations of a plurality of non-protein biomarkers; and assembling a paired training dataset comprising, for each sample, the measured protein biomarker concentration and the measured non-protein biomarker concentrations. In certain embodiments, the paired training dataset is further augmented with physical and chemical sensor condition-monitoring signals obtained from intelligent nanostructures embedded in the non-immunoassay sensors, in accordance with the NEAM approach, providing additional input features that improve model robustness to sensor drift, matrix effects, and environmental perturbations during both training and inference.

In certain embodiments, the invention provides a method of personalizing the machine learning model for an individual subject. The method comprises providing a population-trained machine learning model trained on paired protein and non-protein biomarker measurements from a subject population; obtaining a subject-specific calibration dataset comprising paired protein biomarker and non-protein biomarker measurements from biological fluid samples of the individual subject; and fine-tuning the population-trained machine learning model on the subject-specific calibration dataset using transfer learning to generate a personalized model with improved prediction accuracy for the individual subject. As subject-specific data accumulates over the course of continuous monitoring, the personalized model may be updated iteratively, enabling increasingly precise protein biomarker prediction over time.

In certain embodiments, the invention provides a method of selecting an optimal non-protein biomarker panel for predicting a targeted protein biomarker concentration. The method comprises obtaining a training dataset comprising paired measurements of the targeted protein biomarker and a candidate set of non-protein biomarkers from an ensemble of biological fluid samples; applying a feature importance or feature selection algorithm to the training dataset to rank the candidate non-protein biomarkers by their individual and joint predictive contribution to the targeted protein biomarker concentration; and selecting a subset of the candidate non-protein biomarkers having the highest joint predictive contribution as the non-protein biomarker panel for the trained machine learning model. Suitable feature selection algorithms include without limitation recursive feature elimination, LASSO regularization, mutual information analysis, Shapley additive explanations (SHAP), and permutation importance analysis.

### E. Error Risk Analysis Embodiments

In certain embodiments, the invention provides a method for validating each machine learning-generated prediction of a targeted analyte concentration in a biological fluid, using a module herein termed Error Risk Analysis (ERA). The ERA method comprises generating, by a trained machine learning model, a predicted concentration of a targeted analyte from measured concentrations of a plurality of secondary analytes; calculating, by the ERA algorithm, a risk score for the predicted concentration, wherein the risk score represents an estimated probability that the error in the predicted concentration exceeds a predefined accuracy threshold; and selectively accepting or discarding the predicted concentration based on whether the risk score satisfies a predefined risk criterion. In certain embodiments, calculating the risk score comprises evaluating one or more of: the measured values of the plurality of secondary analytes relative to their expected physiological ranges; the statistical confidence interval of the machine learning model output; sensor operating condition signals obtained from on-chip condition-monitoring structures; and the rate of change of the secondary analyte concentrations over a preceding time window.

In certain embodiments, when the risk score fails to satisfy the predefined risk criterion, the ERA method initiates a corrective action selected from the group consisting of: discarding the predicted concentration and initiating a new measurement cycle; triggering calibration of one or more sensors in the sensor array; alerting a user or clinician of a potentially unreliable measurement; and requesting a confirmatory measurement by an independent method. ERA thereby prevents inaccurate or unreliable predictions from being acted upon in diagnostic or therapeutic decisions, providing an additional layer of safety critical for closed-loop therapeutic applications.

### F. Closed-loop Therapeutic System Embodiments

In certain embodiments, the invention provides a closed-loop therapeutic system for automated delivery of a therapeutic agent guided by a predicted protein biomarker concentration. The system comprises a sensor array configured to continuously measure concentrations of a plurality of non-protein biomarkers in a biological fluid of a subject; a machine learning inference engine configured to generate a predicted concentration of a targeted protein biomarker from the measured non-protein biomarker concentrations; a therapeutic algorithm configured to generate a dosing instruction based on the predicted protein biomarker concentration; and a drug delivery device configured to deliver a therapeutic agent to the subject in accordance with the dosing instruction, wherein the system operates continuously without immunoassay-based protein measurement. The drug delivery device may be co-located with the sensing device—for example, integrated within the same or a contralateral punctal plug in ophthalmic applications—or may be a separate implanted, wearable, or external device.

In a particularly preferred closed-loop embodiment directed to ophthalmic applications, the biological fluid is tear fluid, the targeted protein biomarker is MMP-9, the therapeutic agent is an anti-inflammatory agent, and the drug delivery device is configured for delivery of the anti-inflammatory agent to the ocular surface of the subject. This embodiment enables for the first time a fully automated, closed-loop dry eye disease management system in which continuous MMP-9 prediction from tear non-protein biomarkers drives real-time anti-inflammatory drug delivery without any laboratory testing or clinician intervention. In a further closed-loop embodiment directed to diabetes management, the biological fluid is blood or interstitial fluid, the targeted protein biomarker is insulin or C-peptide, the therapeutic agent is insulin, and the drug delivery device is an insulin pump. In a further closed-loop embodiment directed to inflammatory disease management, the biological fluid is blood or serum, the targeted protein biomarker is an inflammatory cytokine selected from the group consisting of IL-6, TNF-α, and IL-1β, and the therapeutic agent is an immunomodulatory agent.

### G. Multi-protein Simultaneous Prediction Embodiments

In certain embodiments, the invention provides a method for simultaneously predicting the concentrations of a plurality of targeted protein biomarkers in a biological fluid of a subject without directly measuring any of the targeted protein biomarkers. The method comprises measuring, in a sample of biological fluid obtained from the subject, concentrations of a plurality of non-protein biomarkers; and applying a trained multi-output machine learning model to the measured non-protein biomarker concentrations to simultaneously generate predicted concentrations of each of the plurality of targeted protein biomarkers. The trained multi-output machine learning model is trained on a dataset comprising, for each of a plurality of biological fluid samples, measured concentrations of each of the plurality of targeted protein biomarkers and measured concentrations of the plurality of non-protein biomarkers. In certain such embodiments, the plurality of targeted protein biomarkers comprises two or more proteins selected from the group consisting of MMP-9, lactoferrin, lysozyme, IL-1β, IL-6, TNF-α, and lipocalin-1, and the biological fluid is tear fluid. In certain embodiments, the predicted concentrations of the plurality of targeted protein biomarkers are used in combination to generate a multi-protein diagnostic score for assessment of a disease condition, providing richer diagnostic information than any single protein biomarker prediction alone.

### H. Cross-Fluid Inference Embodiments

In certain embodiments, the invention provides a method for predicting a concentration of a targeted protein biomarker in a first biological fluid of a subject from measurements performed on a second, more accessible biological fluid of the same subject. In such cross-fluid inference embodiments, the method comprises measuring concentrations of a plurality of non-protein biomarkers in a sample of the second biological fluid obtained from the subject; and applying a trained cross-fluid machine learning model to the measured non-protein biomarker concentrations to generate a predicted concentration of the targeted protein biomarker in the first biological fluid. The trained cross-fluid machine learning model is trained on a dataset comprising, for each of a plurality of subjects, measured concentrations of the targeted protein biomarker in samples of the first biological fluid paired with measured concentrations of the plurality of non-protein biomarkers in samples of the second biological fluid obtained from the same subjects. This cross-fluid inference approach is particularly valuable where the first biological fluid is difficult or invasive to collect, enabling protein biomarker prediction from a less invasive sampling source.

In a particularly significant cross-fluid inference embodiment directed to neurodegenerative disease, the first biological fluid is cerebrospinal fluid (CSF), the targeted protein biomarker is selected from the group consisting of amyloid-β 42, phosphorylated tau, total tau, and neurofilament light chain, and the second biological fluid is blood or blood plasma. This embodiment has the potential to reduce or eliminate the need for invasive lumbar puncture in the diagnosis and monitoring of Alzheimer's disease and related neurodegenerative conditions by enabling prediction of CSF protein biomarker concentrations from minimally invasive blood measurements. In a further cross-fluid inference embodiment directed to ophthalmic applications, the first biological fluid is tear fluid, the targeted protein biomarker is an ocular inflammatory protein, and the second biological fluid is blood or interstitial fluid, enabling ocular protein prediction from wearable skin-surface sensors.

I. Accurate-under-imperfect-conditions (acuic) Integration Embodiments

In certain embodiments, the method of the present invention is practiced in combination with the Accurate Periocular Assay Under Imperfect Conditions (AcUIC) approach disclosed in co-pending U.S. Provisional Patent Application No. 63/785,970, incorporated herein by reference in its entirety, to enable accurate protein biomarker prediction even when sensor operating conditions deviate from ideal. In such embodiments, the method comprises measuring, using a sensor array, concentrations of a plurality of non-protein biomarkers in a biological fluid sample obtained from a subject; simultaneously acquiring, from one or more condition-monitoring structures integrated in the sensor array, a plurality of sensor condition signals indicative of deviations of sensor operating conditions from ideal conditions; applying a trained machine learning model to both the measured non-protein biomarker concentrations and the sensor condition signals to generate a predicted concentration of the targeted protein biomarker, wherein the trained model was trained on data acquired under a range of both ideal and imperfect sensor conditions; and calculating, by an Error Risk Analysis (ERA) algorithm, a risk score for the predicted concentration based on the sensor condition signals, and discarding the predicted concentration and initiating a new measurement cycle if the risk score exceeds a predefined threshold.

In certain AcUIC integration embodiments, the sensor condition signals acquired by the condition-monitoring structures comprise one or more of: sample temperature, sample volume, sample age, applied electrode voltage and current, sensor baseline drift, and ionic strength of the biological fluid. Each of these condition signals provides information about the degree to which the current measurement conditions deviate from the conditions under which the machine learning model was trained, enabling the ERA algorithm to make a principled estimate of the probability that the current prediction is accurate. In certain embodiments, the sensor array further comprises one or more microfluidic pumps configured to remove stale biological fluid from, and replenish fresh biological fluid to, the sensing elements, thereby actively reducing imperfect conditions arising from sample aging or inadequate sample volume, and lowering the frequency with which ERA discards predictions and initiates new measurement cycles.

The AcUIC integration embodiments described herein represent a complete and self-correcting continuous protein monitoring system, in which imperfect sensor conditions are not merely tolerated but are actively detected, quantified, compensated for in the machine learning prediction, and—when compensation is insufficient—flagged by ERA and resolved through a new measurement cycle. This approach fundamentally distinguishes the present invention from prior art biosensors designed under the assumption of near-ideal operating conditions, which fail when deployed in the complex, variable environments characteristic of implanted or wearable biological fluid sensing.

### IX. Scope and Equivalents

The foregoing description discloses exemplary embodiments of the invention. Modifications and variations of the described embodiments will be apparent to those skilled in the art and are within the scope of the invention as defined by the appended claims. The scope of the invention is not limited to the specific biological fluids, protein biomarkers, non-protein biomarkers, machine learning model architectures, or sensor types described herein. All references cited herein are incorporated by reference in their entireties.

What is claimed is:

1. A method for predicting a concentration of a targeted protein biomarker in a biological fluid of a subject without directly measuring the concentration of the targeted protein biomarker in the biological fluid sample, the method comprising:
measuring, in a sample of biological fluid obtained from the subject, concentrations of a plurality of non-protein biomarkers, wherein the plurality of non-protein biomarkers comprises at least two distinct non-protein analytes; and
applying a trained machine learning model to the measured concentrations of the plurality of non-protein biomarkers to generate a predicted concentration of the targeted protein biomarker,
wherein the trained machine learning model is trained on a dataset comprising, for each of a plurality of biological fluid samples, (i) a measured concentration of the targeted protein biomarker and (ii) measured concentrations of the plurality of non-protein biomarkers.

2. The method of claim 1, wherein the concentration of each of the plurality of non-protein biomarkers is measured repeatedly by a sensor over time, and the trained machine learning model is applied repeatedly to predict the concentration of the targeted protein biomarker at each time point at which the concentrations of the plurality of non-protein biomarkers are measured.

3. The method of claim 1, wherein the plurality of non-protein biomarkers comprises one or more analytes selected from the group consisting of electrolytes, ions, small molecules, metabolites, lipids, fatty acids, and nucleic acids.

4. The method of claim 1, wherein the targeted protein biomarker is an ocular protein biomarker measured in tear fluid, selected from the group consisting of MMP-9, lactoferrin, lysozyme, lipocalin-1, secretory IgA, IL-1β, IL-6, IL-8, TNF-α, clusterin, annexin A1, tear-specific prealbumin, and MMP-3.

5. The method of claim 1, wherein the plurality of non-protein biomarkers comprises at least three analytes selected from the group consisting of Na⁺, K⁺, Cl⁻, Ca²⁺, pH, osmolarity, glucose, lactate, urea, creatinine, and uric acid.

6. The method of claim 1, wherein the plurality of non-protein biomarkers comprises at least one electrolyte ion, at least one small molecule metabolite, and at least one analyte selected from the group consisting of pH and osmolarity.

7. The method of claim 1, wherein the trained machine learning model comprises a model architecture selected from the group consisting of an artificial neural network, a deep neural network, a recurrent neural network, a support vector regression model, a random forest regression model, a gradient boosting model, a Gaussian process regression model, and an ensemble of two or more of the foregoing.

8. The method of claim 1, further comprising:
calculating, by an Error Risk Analysis (ERA) algorithm, a risk score for the predicted concentration representing a probability that a prediction error exceeds a predefined threshold; and
discarding the predicted concentration and initiating a new measurement cycle when the risk score exceeds the predefined threshold.

9. The method of claim 1, wherein the trained machine learning model is a personalized model that is trained or fine-tuned on biological fluid samples obtained from the subject, in addition to population-level training data, to improve prediction accuracy for the individual subject.

10. The method of claim 1, wherein the biological fluid is selected from the group consisting of tear fluid, whole blood, blood serum, blood plasma, urine, saliva, cerebrospinal fluid, sweat, interstitial fluid, synovial fluid, pleural fluid, peritoneal fluid, amniotic fluid, nasal secretions, and combinations thereof.

11. The method of claim 1, wherein the predicted concentration of the targeted protein biomarker is used to generate a dosing instruction for delivery of a therapeutic agent to the subject, and wherein the therapeutic agent is delivered by a drug delivery device in response to the dosing instruction.

12. A system for predicting a concentration of a targeted protein biomarker in a biological fluid without immunoassay-based protein measurement, the system comprising:
a sensor array configured to simultaneously measure concentrations of a plurality of non-protein biomarkers in a biological fluid, wherein the sensor array does not perform immunoassay-based protein capture; and
a machine learning inference engine configured to receive the measured concentrations of the plurality of non-protein biomarkers and to output a predicted concentration of a targeted protein biomarker,
wherein the machine learning inference engine executes a trained machine learning model trained on paired measurements of the targeted protein biomarker concentration and the plurality of non-protein biomarker concentrations obtained from an ensemble of biological fluid samples.

13. The system of claim 12, wherein the sensor array comprises at least one ion-selective electrode (ISE) sensor, at least one enzymatic electrochemical sensor, and at least one sensor selected from the group consisting of a pH sensor and a conductometric osmometer.

14. The system of claim 12, wherein the sensor array is integrated within a miniaturized implantable or wearable device selected from the group consisting of a punctal plug configured for implantation in a lacrimal punctum, a subcutaneous implant, and a microneedle patch.

15. The system of claim 12, wherein the machine learning inference engine executes on a processor selected from the group consisting of an integrated circuit chip mounted on the sensor array device, an edge computing device in wireless communication with the sensor array, and a cloud computing platform.

16. The system of claim 12, further comprising a drug delivery device configured to receive a dosing instruction generated based on the predicted protein biomarker concentration and to deliver a therapeutic agent to the subject in response to the dosing instruction, forming a closed-loop protein-guided therapeutic system.

17. A method for continuously monitoring a concentration of a targeted protein biomarker in a biological fluid of a subject over a monitoring period, the method comprising:
continuously or repeatedly measuring, using a sensor array in contact with or receiving a biological fluid from the subject, concentrations of a plurality of non-protein biomarkers at a plurality of time points during the monitoring period;
applying, at each of the plurality of time points, a trained machine learning model to the measured non-protein biomarker concentrations to generate a predicted concentration of the targeted protein biomarker; and
generating a longitudinal profile of predicted targeted protein biomarker concentration over the monitoring period,
wherein the sensor array does not perform immunoassay-based protein measurement, and wherein continuous monitoring is enabled by the absence of antibody-antigen reagent consumption in the non-protein biomarker sensing steps.

18. The method of claim 17, wherein the monitoring period is at least 24 hours and the non-protein biomarker concentrations are measured at intervals of one hour or less.

19. The method of claim 17, wherein a change in the predicted concentration of the targeted protein biomarker over the monitoring period is used to assess at least one of disease progression, therapeutic response, and need for clinical intervention.

20. The method of claim 17, wherein the biological fluid is tear fluid and the sensor array is embedded in a punctal plug implanted in a lacrimal punctum of an eye of the subject, enabling continuous, implanted monitoring of tear protein biomarker concentrations.
